(19) United States
(12) Patent Application Publication
(10) Pub. No.: US 2015/0050648 A1
(43) Pub. Date: Feb. 19, 2015

(54) Synthetic DNA-Antibody Complex as External Reference for Chromatin Immunoprecipitation

(71) Applicants: Neus VISA, Bandhagen (SE); Ann-Kristin ÖSTLUND FARRANTAS, Farsta (SE); Stefanie Gabriele BÖHM, Solna (SE); Andrea EBERLE, Solna (SE)

(72) Inventors: Neus Visa, Bandhagen (SE); Ann-Kristin Östlund Farrantas, Farsta (SE); Stefanie Gabriele Böhm, Solna (SE); Andrea Eberle, Solna (SE)

(21) Appl. No.: 14/356,528

(22) PCT Filed: Nov. 9, 2012
(86) PCT No.: PCT/SE2012/051224

§ 371 Date: May 6, 2014

(30) Foreign Application Priority Data
Nov. 10, 2011 (SE) 1151061-7

(51) Int. Cl. C12Q 1/68

(52) U.S. Cl. CPC C12Q 1/6806
USPC 435/6.11; 530/391.3

(57) ABSTRACT

The present invention provides an external standard in the form of a nucleic acid-antibody complex to be used in a chromatin immunoprecipitation method.

The present invention is in the field of chromatin immunoprecipitation (ChIP). More particularly, the invention is directed to an external standard to be used in a ChiP in order to more reliably being able to detect and quantify interactions between a protein of interest and genomic DNA.

### BACKGROUND OF THE INVENTION

Research in many areas of molecular cell biology often relies on the use of quantitative analytical methods that require assessments of reproducibility and statistical significance. A very powerful quantitative method that has become widely used for studies of gene expression and epigenetics is chromatin immunoprecipitation (ChIP). ChIP detects and quantifies interactions between a protein of interest and genomic DNA in vivo (reviewed by Aparicio et al., 2004). A typical application of ChIP is the mapping of the binding sites of individual DNA-binding proteins (for example post-translationally modified histones, transcription factors, or chromatin remodeling factors) either genome-wide or within specific genomic areas. The knowledge of the proteins associated to a specific genomic region and the characterization of the post-translational state of the histones in that region provide insight into the function and activity of the genomic region of interest. Proteins bound to RNA co-transcriptionally, for instance splicing factors or mRNA-binding proteins, can also be analyzed by ChIP (Gilbert and Svejstrup, 2006; Listerman et al., 2006). This is a relevant application of ChIP because many pre-mRNA processing reactions take place at the gene and are influenced by the structure of the chromatin.

In a classic ChIP experiment (FIG. 1), tissues or cultured cells are fixed with formaldehyde to make covalent bindings between proteins and nucleic acids in the cell nucleus, a process called cross-linking. After harvesting the cross-linked cells, the chromatin is extracted and fractionated, usually by sonication, to obtain fragments of approximately 500 bp or less. Shorter chromatin fragments provide better resolution when determining the exact binding sites of a specific protein. A specific antibody is used to immunoprecipitate the cross-linked protein/DNA complexes, and the bound fraction is isolated using protein A and/or protein G coupled to either Sepharose or magnetic beads. Next, the proteins are degraded, the cross-linking reversed and the DNA purified. Real-time PCR (quantitative PCR, qPCR) is often used to quantify the immunoprecipitated DNA and in this way assess the amount of protein bound to the genomic regions of interest in vivo. Alternatively, the ChIP method can be used to map the distribution of a protein in the entire genome. In this case, the immunoprecipitated DNA is analyzed by microarray hybridization (ChIP-chip method) or directly sequenced (ChIP-seq method) (reviewed by Barski and Zhao, 2009).

Many technical aspects are critical when carrying out quantitative ChIP experiments and a common problem is the difficulty in reproducing results in a quantitative manner. The biological variability can be minimized by standardizing the quality and the handling of the samples. Monitoring the yields of recovery in the subsequent steps of the process is a more difficult task. Variations in the efficiency of the immunoprecipitation and losses of material during the purification of the DNA are major factors that contribute to the variability of the results. These variations are reflected in the statistics of the data and reduce the accuracy of the measurements. In other analytical methods, for instance measurements of gene expression levels, the use of an internal reference for normalization of the data is a common practice. However, in most ChIP experiments it is not known which genes are affected by a given experimental treatment (for example treatment with a drug, depletion or over-expression of a protein), which precludes the use of suitable internal references.

### SUMMARY OF INVENTION

The present inventors have found that the use of an external reference in a chromatin immunoprecipitation method may be used to at least mitigate the above mentioned limitations. The present document therefore provides an external standard in the form of a nucleic acid-antibody complex to be used as an external standard in a chromatin immunoprecipitation method.

Disclosed is therefore the use of a synthetic nucleic acid-antibody complex as an external reference in a chromatin immunoprecipitation method. Said nucleic acid in the nucleic acid-antibody complex may e.g. be DNA, RNA, DNA-RNA hybrid, locked nucleic acid or peptide nucleic acid. The nucleic acid may e.g. be labeled with digoxigenin, biotin and/or fluorescein isothiocyanate. The nucleic acid-antibody complex may be formed by labeling a nucleic acid with a label, said label being recognizable by an antibody, cross-linking said labeled nucleic acid to an antibody recognizing said label, thereby forming said nucleic acid-antibody complex. The cross-linking may be performed by using formaldehyde or 3′-dithiobispropionimidate. The nucleic acid-antibody complex may be added to a cross-linked chromatin sample to be analyzed by chromatin immunoprecipitation before carrying out the chromatin immunoprecipitation method.

Also disclosed is a method for preparing a nucleic acid-antibody complex as defined in this document, said method comprising the steps of:

a) providing a nucleic acid fragment
b) label said nucleic acid fragment with a label to provide a labeled nucleic acid
c) contacting said labeled nucleic acid of step b) with an antibody recognizing said label
d) cross-linking said antibody to said labeled nucleic acid to form a cross-linked antibody-nucleic acid complex.

Further disclosed is a chromatin immunoprecipitation method comprising the steps of:

a) providing a cross-linked chromatin sample to be analyzed
b) adding a synthetic nucleic acid-antibody complex as defined in this document to the chromatin sample of step a)
c) performing an immunoprecipitation in the presence of an antibody recognizing a putative protein, such as histone, in the chromatin sample of step a)
d) reversing the cross-linking of said cross-linked chromatin sample and said nucleic acid-antibody complex
e) purifying the DNA and nucleic acid obtained in step d); and
f) quantifying the amount of said DNA and said nucleic acid obtained in step e).

This document is also directed to a kit of parts for use as an external standard in a chromatin immunoprecipitation method, said kit comprising:

a) a nucleic acid-antibody complex as defined herein
b) a pair of primers to quantify the nucleic acid in the nucleic acid complex of a).

Further disclosed is a kit of parts for performing a chromatin immunoprecipitation method of, said kit comprising, in addition to reagents necessary to perform a regular chromatin immunoprecipitation method, a nucleic acid-antibody complex as defined herein and a pair of primers to quantify the nucleic acid in the nucleic acid complex.

Also disclosed is a kit of parts for performing a chromatin immunoprecipitation method, said kit comprising:

a) cell resuspension buffer
b) glycine solution
c) lysis and extraction buffers
d) chromatin shearing buffer
e) supplementary detergent solution for immunoprecipitation buffer
f) washing buffer
g) protease inhibitor cocktail
h) pre-clearing beads
i) pre-blocked Protein A or Protein G beads
j) reversal solution
k) DNA purification system
l) external standard reagent
m) a nucleic acid-antibody complex as defined in any one of claims 1-7
n) optionally PCR primers for quantification of the external standard.

### FIGURE LEGENDS

FIG. 1: Schematic overview of a ChIP experiment using the DNA-DIG-antibody complex as an external reference. The genomic DNA is shown as a black line; the different proteins are drawn as stars, hexagons and rectangles. The DNA labeled with DIG is shown in grey and is bound by the anti-DIG antibody. The beads illustrate the protein A/G Sepharose or magnetic beads. See the main text for explanations.

FIG. 2: Immunoprecipitation and stability of the DNA-DIG-antibody complex.

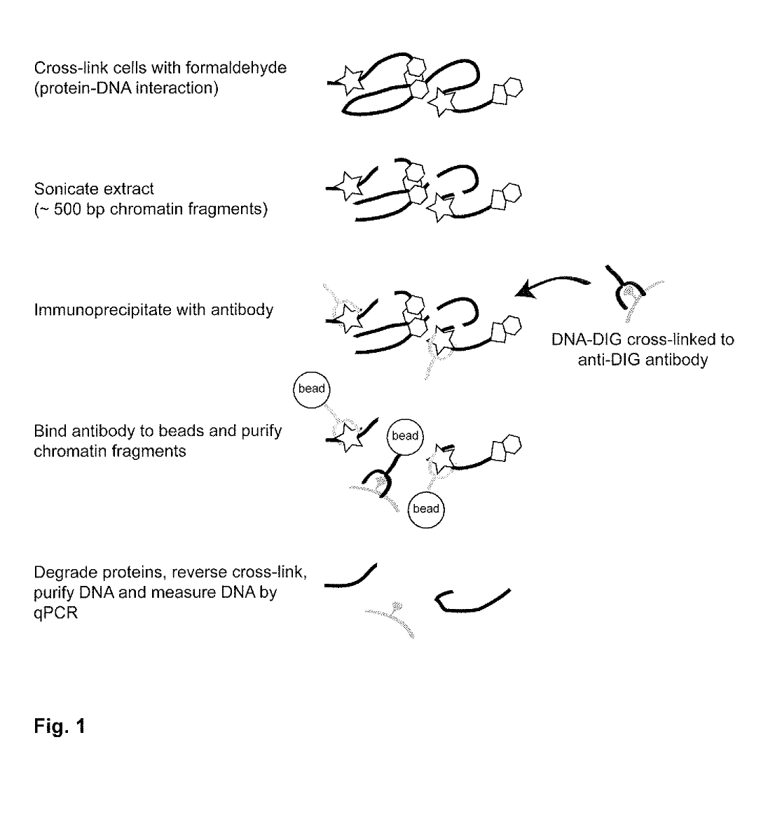

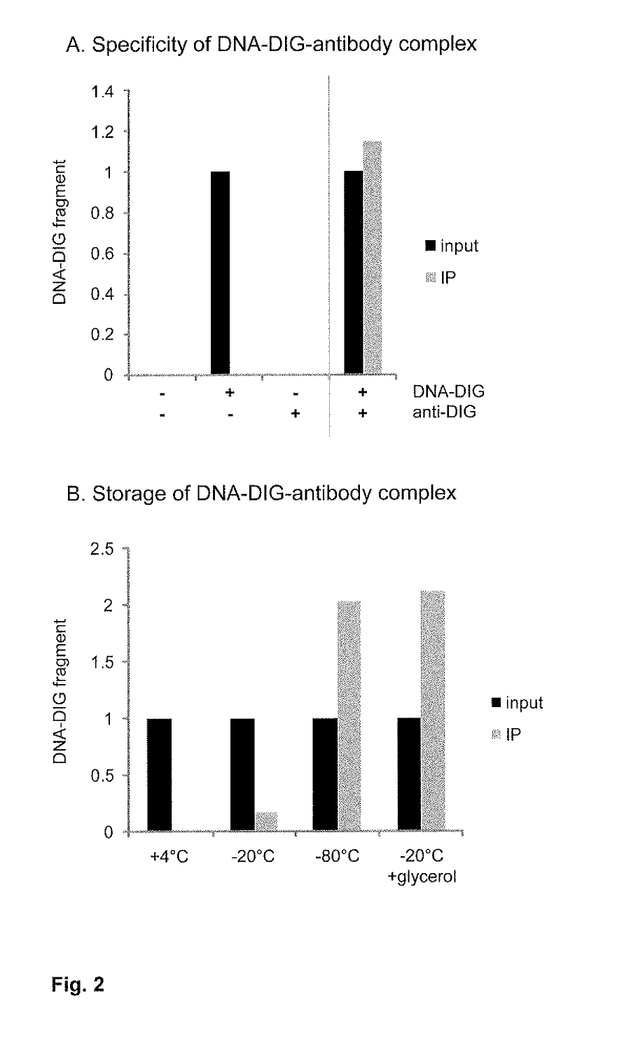

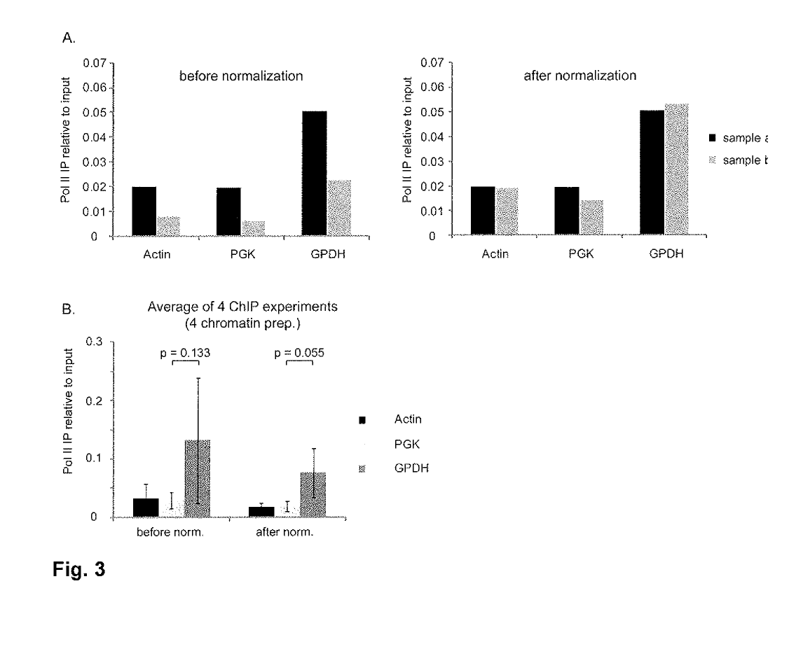

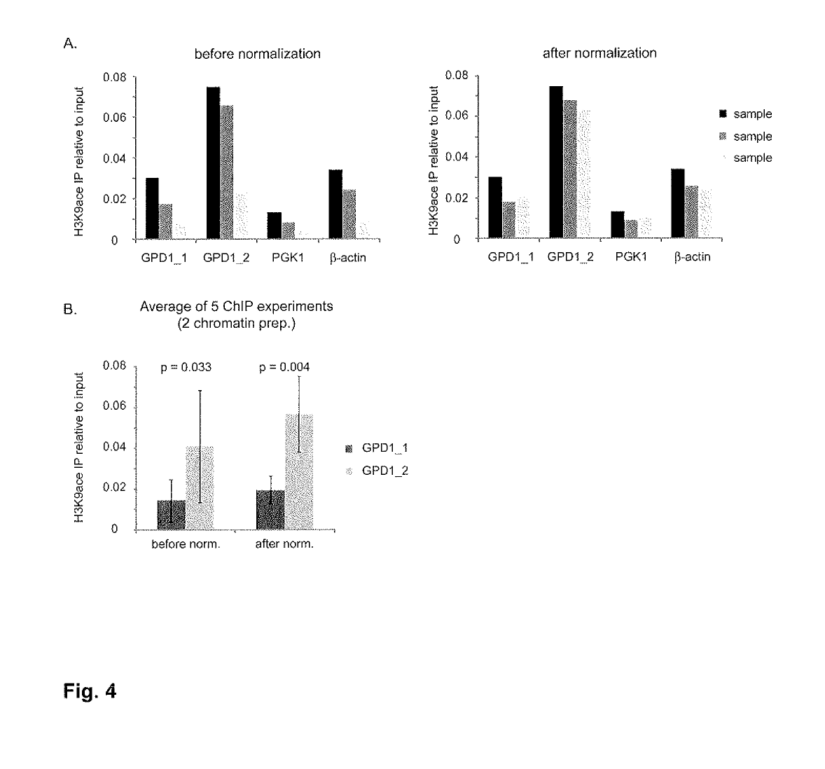

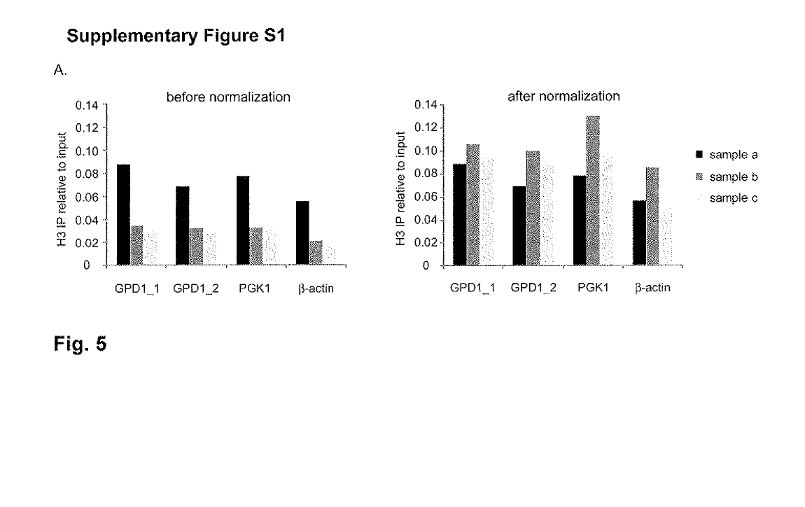

A. Specific immunoprecipitation of the DNA-DIG-antibody complex. The DNA-DIG-antibody complex was added to a chromatin extract prepared from Drosophila S2 cells and the ChIP experiment was performed as shown in FIG. 1 but omitting the specific antibody. The DNA-DIG in the input (using 1/100 of the total amount of starting material) and the immunoprecipitated DNA-DIG (IP) were quantified by two qPCR runs, each in duplicate. The histogram shows the average amount of immunoprecipitated DNA relative to the amount of DNA present in the input (for the DNA-DIG and for the DNA-DIG-antibody complex). The signals of the samples with chromatin alone and with anti-DIG antibody alone are set relative to the input signal of the DNA-DIG sample. B. Storage of the DNA-DIG-antibody complex. The DNA-DIG-antibody complex was stored under different conditions as indicated in the figure and used in a ChIP experiment as in FIG. 2A. The average signals of the ChIP sample measured by two qPCR runs with duplicates are shown relative to the corresponding input signal.

FIG. 3: The use of the DNA-DIG-antibody complex as external reference in ChIP experiments with Drosophila chromatin.

A. The DNA-DIG-antibody complex compensates for losses of material. Chromatin was prepared from D. melanogaster S2 cells and supplemented with the DNA-DIG-antibody complex. The chromatin extract was divided in two parts (sample a and sample b) that were used for ChIP using an antibody against the Pol II. The samples were treated in parallel, but in sample b half of the immunoprecipitated material was discarded before the DNA purification (see the main text for details). The abundance of actin, PGK, and GPDH sequences in the immunoprecipitated DNA were quantified by qPCR. Each sample was quantified in two independent qPCR runs, each in duplicate. The left panel shows the average signals for each sample relative to the input. The levels of DNA detected in sample b are lower, as expected. The DNA-DIG fragment was quantified in parallel. The right panel shows the average signals obtained for each gene relative to the input after normalization with the levels of DNA-DIG measured in each sample. The results obtained for samples a and b are much more similar to each other after normalization.
B. The use of the DNA-DIG-antibody complex to normalize ChIP experiments using different chromatin batches. Four ChIP experiments with antibodies against Pol II were performed using four different chromatin batches. The immunoprecipitated DNA was measured in two qPCR runs, each in duplicate. The levels of actin, PGK and GPDH are shown relative to the corresponding input sample (left panel, before normalization) and relative to the input and normalized to the DNA-DIG fragment (right panel, after normalization). Using a paired, two-tailed Student's t-test, the p-value between PGK and GPDH was calculated before normalization (p=0.133) and after normalization (0.055) (n=4).

FIG. 4: The use of the DNA-DIG-antibody complex as external reference in ChIP experiments with human chromatin.

A. The DNA-DIG-antibody complex can compensate for losses of material. Chromatin was prepared from HeLa cells and supplemented with DNA-DIG-antibody complex. The supplemented chromatin extract was then split into three parts (sample a, sample b and sample c) that were processed in parallel as in FIG. 3. The ChIP was carried out using an antibody against histone H3 acetylated in lysine 9 (anti-H3K9ace). In samples b and c, part of the material was discarded before purification of the DNA. The abundances of GPD1, PGK1, and β-actin sequences in the immunoprecipitated DNA were quantified by qPCR and are shown relative to the input. For GPD1, two primer pairs were used: one specific for the promoter region (GPD1_{—}1) and a second one for the coding region (GPD1_{—}2). The DNA-DIG fragment in each sample was also quantified in parallel by qPCR and used for normalization, as in FIG. 3A. Note that the signals obtained for samples b and c are much more similar to sample a after normalization. B. The use of the DNA-DIG-antibody complex to normalize ChIP experiments using different chromatin batches. Five independent ChIP experiments using chromatin from two different batches were carried out. The levels for GPD1 in the promoter region (GPD1_{—}1) and in the coding region (GPD1_{—}2) were quantified by qPCR in the input and in the immunoprecipitated DNA. The average levels are shown relative to the corresponding input sample. The left panel shows the results obtained before normalization. The right panel shows the results when the DNA-DIG abundance was used for normalization. A paired, two-tailed Students t-test, was used to compare the densities of H3K9ace in the two regions analyzed. The resulting p-values were p=0.033 before normalization and p=0.004 after normalization (n=5).

FIG. 5: The use of the DNA-DIG-antibody as external reference in a ChIP experiment with an antibody against histone H3.

Chromatin was prepared from HeLa cells and supplemented with DNA-DIG-antibody complex. The supplemented chromatin extract was then split into three parts (sample a, sample b and sample c) that were processed in parallel. The ChIP was carried out as described in Materials and Methods using an antibody against histone H3 (anti-H3). In samples b and c, part of the material was intentionally discarded before purification of the DNA to mimic losses of material. The abundances of GPD1, PGK1, and β-actin sequences in the immunoprecipitated DNA were quantified by qPCR and are shown relative to the input. The left panel shows the average signals for each sample relative to the input. The levels of DNA detected in samples b and c are lower than those of sample a, as expected. The DNA-DIG fragment was quantified in parallel and the right panel shows the average signals obtained for each of the genes relative to the input after normalization with the levels of DNA-DIG measured in each sample.

### DETAILED DESCRIPTION OF THE INVENTION

Chromatin immunoprecipitation (ChIP) is an analytical method used to investigate the interactions between proteins and DNA in vivo. Variations in the efficiency of the immunoprecipitation and losses of material during the purification of the DNA are sources of variability that reduce the accuracy of the results and impair the use of ChIP as a quantitative tool. We have developed a simple method to improve the quantification of ChIP data based on the use of an external reference. At the core of this method is a synthetic DNA-antibody complex that is treated with formaldehyde to mimic the behavior of cross-linked chromatin. The rationale of the method is that this DNA-antibody complex, once added to the chromatin extract, will undergo the same treatments as the rest of the sample, including immunopurification, reversal of the cross-linking, purification of the DNA and quantification of the recovered DNA. A fixed amount of this synthetic DNA-antibody complex is spiked into the chromatin extract at the beginning of the ChIP experiment. The amounts of synthetic DNA recovered in each tube are measured at the end of the process and used to normalize the results obtained with the antibodies of interest. We have chosen to use a DNA of bacterial origin without homology with eukaryotic sequences, we have labeled this DNA with digoxigenin (DIG) and we have used an anti-DIG antibody to form a DNA-DIG-complex. Using this DNA-DIG-antibody complex as an external reference, we could strongly reduce the variability between individual ChIP samples, which increased the accuracy and the statistical resolution of the data.

As mentioned above, in most ChIP experiments it is not known which genes are affected by a given experimental treatment (for example treatment with a drug, depletion or over-expression of a protein), which precludes the use of suitable internal references. To circumvent this limitation, we propose the use of a synthetic, exogenous normalization probe that is added in a constant amount to every single sample before the immunoprecipitation. We have designed a synthetic probe containing a bacterial DNA sequence that lacks homology to eukaryotic genomes. We have labeled the DNA with digoxigenin (DIG), cross-linked the DNA-DIG to an anti-DIG antibody, and used the DNA-DIG-antibody complex as external reference in ChIP experiments. The rationale of the method is that the synthetic probe, once added to the chromatin extract, will undergo the same treatments as the rest of the sample, including immunopurification, reversal of the crosslinking, purification of the DNA and quantification of the recovered DNA by qPCR. At the beginning of the ChIP experiment, the same amount of the synthetic DNA-DIG probe is spiked into each tube and differences in the amounts of DNA-DIG recovered can be used to compensate for differences in the recovery yields among individual tubes. Using this normalization tool, we could strongly reduce the variability between the individual ChIP samples which in turn increased the statistical resolution of the data.

The external reference nucleic acid-antibody complex can contain any type of nucleic acid fragment provided that the sequence of the fragment does not have any significant homology to any sequences present in the chromatin to be analyzed by the ChiP, and provided that the nucleic acid can be quantified efficiently. Examples of alternative sequences to DNA are RNA, DNA-RNA hybrid, locked nucleic acids (LNAs) or peptide nucleic acids (PNAs). Examples of sequences to be used for the nucleic acid include, but are not limited to, sequences from E. coli, sequences from other microorganisms, and/or synthetic sequences.

The length of the nucleic acid fragment in the external reference nucleic acid-antibody complex can vary. Longer nucleic acid molecules (in the range of several hundred bp) might be advantageous because they might better mimic the behavior of fixed chromatin in a typical ChIP experiment.

The labeling of the nucleic acid in the external reference nucleic acid-antibody complex is to facilitate the binding of an antibody to the nucleic acid. The nucleic acid can be labeled with a label such as with DIG or with other small molecules provided that there is a specific antibody directed against the label of choice and provided that the antibody does not cross-react with any antigen in the chromatin used for the experiment. Examples of alternative labels are biotin and fluorescein isothiocyanate (FITC).

Any cross-linking method can be used to cross-link the nucleic acid-antibody complex in the external reference nucleic acid-antibody complex provided that the cross-linking is reversed in the conditions of the ChIP experiment. Examples of suitable cross-linking agents include, but are not limited to formaldehyde and 3′-dithiobispropionimidate (DTBP). Preferably, the same cross-linking agent is used for cross-linking the sample to be analyzed and the external reference nucleic acid-antibody complex.

### Experimental Section

### Material and Methods

### PCR and DIG Labeling

For generating the DNA fragment, a PCR was performed with the Taq polymerase (Fermentas) using 10 ng plasmid encoding the quinol bo3 oxidase (Frericks et al., 2006), 0.4 μM forward primer 5′-GTGCGCGAACGTACTGATTA-3′ and reverse primer 5′-AGATAGCGATCCAGGGTCAA-3′.

DIG labeling of the DNA was performed by PCR using the same primers specified above in the presence of Digoxigenin-11-dUTP (Roche). The reaction contained 0.2 mM dATP, 0.2 mM dGTP, 0.2 mM dCTP, 0.18 mM dTTP and 0.01 mM DIG-11-dUTP.

### Preparation and Purification of DNA-DIG-Antibody Complex

The DNA-DIG product was purified using “illustra GFX™ PCR DNA and Gel Bad Purification Kit” (GE Healthcare) and incubated together with mouse monoclonal anti-digoxigenin antibodies (Abeam, ab420) for 2 hours at room temperature. The amount of the antibody was calculated that theoretically each binding site of the antibody interacts with one DIG molecule of the DNA fragment. DNA-DIG and anti-DIG antibody were cross-linked by addition of formaldehyde to a final concentration of 2% for 10 minutes. The cross-linking was terminated by addition of glycine to a final concentration of 0.125 M and incubation for 10 minutes. The cross-linked DNA-DIG-antibody complex was finally purified in Nanosep 100K columns using PBS buffer.

Quantitative PCR (qPCR)

For real-time PCR (qPCR), immunoprecipitated DNA was amplified in 20 μl KAPA SYBR Fast qPCR Master Mix (KAPA Biosystems) using the RotorGene (Qiagen). The sequences of all primers used can be found in table 1.

TABLE 1
list of oligonucleotides used for qPCR

| | Forward primer | Reverse primer |
|---|---|---|
| NP-F, NP-R | 5′-tattgcttccttccc | 5′-gtcaacaacgcgacg |
| (DNA-DIG) | aattctg-3′ | gtaa-3′ |
| Actin | 5′-gcacacccacaagct | 5′-ttgcgctttgggaaa |
| | tacaca-3′ | tatcttc-3′ |
| GPDH | 5′-aatcgcggagccaag | 5′-agcccacaatgcaca |
| | tagta-3′ | cattt-3′ |
| PGK | 5′-gctcaccgacaaaat | 5′-ggatacttcctgtgc |
| | gacct-3′ | gtgct-3′ |
| GPD1_1 | 5′-ctccccacccaccca | 5′-ggggcctacccttcc |
| | tggag-3′ | cccat-3′ |
| GPD1_2 | 5′-cgccagcaccctctt | 5′-taccctggccggtct |
| | tgggg-3′ | ggagc-3′ |
| PGK1 | 5′-gtggggcagcagcag | 5′-tgggaggaatgggct |
| | tggag-3′ | ggggc-3′ |
| β-actin | 5′-ggacttcgagcaaga | 5′-agcactgtgttggcg |
| | gatgg-3′ | tac-3′ |

### Chromatin Immunoprecipitation (ChIP)

ChiP was carried out essentially as described by Takahashi et al. (2000). The cells were fixed at room temperature for 10 min by the addition of a fixing solution containing formaldehyde (final concentration 2%). After incubation with 0.1 M glycine for 10 min, the cells were spun down at 500 g for 5 min. The pellet was resuspended in cold buffer 1 (50 mM Hepes, pH 7.6, 140 mM NaCl, 1 mM EDTA, 10% glycerol, 0.5% NP-40, 0.25% Triton-X, Complete protease inhibitors (Roche)) and incubated 10 min at 4° C. The cells were spun down again as above. After centrifugation, the cells were resuspended in buffer 2 (10 mM Tris, pH 8, 200 mM NaCl, 1 mM EDTA, 0.5 mM EGTA, protease inhibitors) and incubated at room temperature for 10 min. The samples were centrifuged and the pellet resuspended in buffer 3 (10 mM Tris, pH 8, 1 mM EDTA, 0.5 mM EGTA, protease inhibitors). The obtained chromatin was sheared by sonication to give a DNA size of 250-900 bp. After centrifugation at 16 000 g for 30 min at 4° C., the samples were pre-cleared with Sepharose A/G slurry for 2 hours on a rotating wheel. Immunoprecipitation was performed over-night at 4° C. with primary antibody in the presence of 0.1% deoxycholic acid and 1% triton. Blocked A/G slurry was added and incubation was prolonged for one hour. After incubation, the samples were spun down and washed 5 times 10 minutes each with RIPA buffer (50 mM Hepes, pH 7.6, 500 mM NaCl, 1 mM EDTA, 1% NP-40, 0.8% deoxycholic acid). The last washing step was performed with 50 mM Tris, pH 8 and 2 mM EDTA. The pelleted beads was then resuspended in TE-Buffer supplemented with SDS, RNAse A and proteinase K for 3 hours at 55° C. and over-night at 65° C. The DNA was purified with phenol:chloroform and precipitated with ethanol. The amounts of immunoprecipitated DNA were quantified by qPCR.

The antibodies used for ChIP were from Abcam (ab5408 against Pol-II, ab1791 against histone H3 and ab10812 against H3K9ace).

### Results and Discussion

### Preparation of a Synthetic DNA-DIG-Antibody Complex

We wanted to design a DNA-antibody complex that could be used as external reference in ChIP experiments with samples from different origins. It was important to choose a DNA sequence that could be mixed with the chromatin extract used for ChIP, immunoprecipitated, and quantified without interfering with the ChIP experiment itself. For this reason it was important to choose a DNA sequence not present in the chromatin extract. We chose fly and human as reference systems for the development of the method. We reasoned that a DNA sequence of bacterial origin that is not found in eukaryotic genomes could serve this purpose and we chose a 78-bp long sequence belonging to the bo3 oxidase operon (cyo) of Escherichia coli. This sequence was not found in eukaryotes, as shown by Blastn and Megablast homology analyses.

It was also important to choose a sequence that could be quantified accurately. To test whether the quinol bo3 oxidase sequence could be quantified accurately by qPCR, we amplified the 78-bp long DNA fragment from a plasmid encoding the quinol bo3 oxidase (Frericks et al., 2006). We used the 78-bp long PCR product as a template in a qPCR reaction with nested primers (NP-F and NP-R) to amplify an amplicon of 45 bp (Table 1). The melting curve and the reaction efficiency of the qPCR reaction were monitored and found to be satisfactory (efficiency=1, R=0.99984). Since we wanted to use the DNA fragment as an external reference in ChIP experiments involving DNA of animal origin, it was crucial that the primers in the qPCR would not amplify any DNA sequence from the organisms of origin. Therefore, qPCR reactions were performed using genomic DNA from either human or Drosophila melanogaster as templates. No signals above background were obtained (data not shown).

After these initial control experiments, we proceeded to use the 78 nt DNA fragment to make an DNA-antibody complex that could serve as external reference for ChIP. To this aim, the 78-bp DNA product was labeled by PCR using DIG-11-dUTP that was incorporated in the product instead of dTTP. A ratio between dTTP and the DIG-labeled dUTP was chosen in the PCR reaction to obtain an average incorporation of 1.4 DIG-11-dUTPs per DNA molecule. The resulting DNA-DIG product was purified and bound to an anti-DIG antibody. The DNA-DIG-antibody complex was cross-linked with formaldehyde and purified as described in Materials and Methods. DNA-antibody complexes can be stabilized by other means but we wanted to produce a DNA-antibody complex that would mimic the cross-linked DNA-protein complexes in the chromatin. Therefore we chose to use the same type of cross-linking that is used to prepare chromatin in typical ChIP protocols.

### Analysis of Specificity, Storage, and Concentration of the DNA-DIG-Antibody Complex

In the next series of experiments, we investigated whether the DNA-DIG-antibody complex could be immunoprecipitated and quantified in the conditions of a ChIP experiment. Therefore the DNA-DIG-antibody complex was added to a chromatin extract prepared from D. melanogastor S2 cells, and immunoprecipitated using protein A/G-Sepharose beads following a conventional ChIP protocol but without any other antibody present in the immunoprecipitation mixture. Control reactions lacking either DNA-DIG and/or anti-DIG antibody were run in parallel to assess the specificity of the purification (FIG. 2A). After the entire ChIP procedure, the amount of immunoprecipitated DNA-DIG was quantified in each sample and in the initial chromatin extract (referred to as input). The quantification was carried out by qPCR using the NP-F and NP-R primers. The qPCR results were expressed relative to the levels present in the input. The DNA-DIG was immunoprecipitated efficiently only when the DNA-DIG-antibody complex was present in the immunoprecipitation mixture. Addition of DNA-DIG alone did not result in any recovery, as expected. The anti-DIG antibody alone did not lead to a detectable signal either (FIG. 2A).

In another series of experiments, we tested the stability of the DNA-DIG-antibody complex. To assess the effect of the storage conditions on the performance of the complex, we carried out immunoprecipitation experiments like the one described above with DNA-DIG-antibody complexes that had been stored for 4 weeks at different temperatures: +4, −20 or −80° C. We also tested the effect of the presence of glycerol on the stability of the complex. Complexes stored at 4° C. or at −20° C. in the absence of glycerol could not be efficiently immunoprecipitated (FIG. 2B). The input signals were similar in all samples indicating that the DNA was not degraded. Therefore, the problem was either the immunoreactivity of the antibody or the stability of the cross-linking between the DNA-DIG and the anti-DIG antibody (FIG. 2B and data not shown). From this experiment we concluded that the DNA-DIG-antibody complex should be stored at −80° C. or at −20° C. in a buffer containing glycerol.

To apply the DNA-DIG-antibody complex as a tool for normalizing ChIP data, it is important to titrate the amount of DNA-DIG-antibody complex used. For optimal performance, the cycle threshold (Ct) values obtained for the DNA-DIG should be in the same range as the Ct values obtained for the gene of interest in the ChIP experiment (data not shown).

Normalization of ChIP Experiments with Drosophila Chromatin

We carried out test experiments aimed at validating the usefulness of the DNA-DIG-antibody complex to compensate for losses of material in ChIP experiments. ChIP protocols are composed of many different steps that all can contribute to variation in the amount of DNA recovered. The chromatin was prepared from D. melanogaster S2 cells and two independent ChIP experiments were performed from the same starting material (referred to sample a and sample b in FIG. 3A). In both cases, the chromatin was immunoprecipitated with an antibody directed against the C-terminal domain (CTD) of the RNA polymerase II. To mimic a situation in which part of the sample is lost during the experiment, only half of the immunoprecipitated DNA was purified and precipitated in sample b. The other half was discarded. After all steps in the ChIP procedure, the relative amounts of three endogenous housekeeping genes—actin, PGK, and GPDH—were quantified by qPCR. The DNA-DIG fragment was also quantified in parallel. As shown in FIG. 3A, the signals from the two ChIP samples (sample a and sample b) from the same starting material differed strongly without normalization (FIG. 3A, left panel). By normalizing the values of actin, PGK, and GPDH with the amount of DNA-DIG fragment recovered, the outcome of the two ChIP samples became very similar (FIG. 3A, right panel). We therefore concluded that the DNA-DIG-antibody complex is a useful tool for normalization of ChIP data and can compensate for losses of material.

In the experiment reported above, the DNA-DIG-antibody complex could correct variations that arise after chromatin preparation, such as efficiency of immunoprecipitation, loss of beads, and DNA purification. In a next experiment we tested whether the DNA-DIG-antibody complex could also improve the compilation of ChIP data derived from different chromatin preparations. Four independent ChIP reactions using four different chromatin preparations were performed and analyzed as explained above. FIG. 3B shows the results obtained for the three housekeeping genes. The results are expressed as average of the four experiments before and after normalization with the DNA-DIG-antibody complex. The normalization could compensate for individual variations and therefore gave a pronounced reduction of the standard deviations, which increased the accuracy of the measurements and revealed biological differences to a better extent. For instance, a student's T-test comparison of the signals obtained for GPDH and PGK did not give any significant difference between the two genes prior to normalization (p-value=0.133) but revealed more significant differences when the values were normalized against the DNA-DIG signal (p-value=0.055). In summary, this result indicates that the variations between the independent samples were reduced when using the DNA-DIG-antibody complex as a normalization tool. There was still some variability left as the method cannot compensate for some of the sources of variation, such as differences in the quality of the different chromatin extracts.

Normalization of ChIP Experiments with Human Chromatin

We also performed ChIP experiments with chromatin isolated from human HeLa cells. The chromatin was immunoprecipitated by antibodies against histone H3 acetylated in lysine 9 (anti-H3K9ace, FIG. 4) or against histone H3 (anti-H3, FIG. S1). Three independent ChIP reactions (referred to as samples a, sample b, and sample c) were performed from the same chromatin batch and the housekeeping genes GPD1, PGK1, and actin were measured by qPCR (FIGS. 4A and S1). Two regions in the GPD1 gene were analyzed, as indicated in the figures. As in the experiment shown in FIG. 3A, part of the immunoprecipitated DNA was deliberately discarded in two of the samples to generate variability and assess the ability of the DNA-DIG-antibody complex to compensate for losses of material. The comparisons of the results obtained before and after normalization of the data against the DNA-DIG reference shows that the DNA-DIG-antibody complex also reduces the unevenness among the three samples when human chromatin is used (compare left and right panels in FIGS. 4A and S1).

Next, we carried out five independent ChIP reactions using chromatin from two different preparations. Similar to the ChIP experiments done with Drosophila chromatin, the handling of different chromatin batches leads to a larger variation between different samples. The difference in the abundance of H3K9ace in the promoter (GPD1_{—}1) and coding region (GPD1_{—}2) of the GPD1 gene was more significant when the DNA-DIG-antibody normalization was applied. The DNA-DIG normalization improved the statistical significance of the comparison and shifted the p value from 0.033 to 0.004 (FIG. 4B). We concluded that the use of the DNA-DIG-antibody complex compensates for variations between independent ChIP samples, even when the individual samples come from different chromatin batches.

### CONCLUDING REMARKS

We have developed a simple method to improve the quantification of ChIP results based on the use of a DNA-DIG-antibody complex that works as an external reference for normalization purposes. We show that the use of the DNA-DIG-antibody increases the accuracy of the measurements as illustrated by the pronounced reduction of the standard deviations obtained in two independent series of experiments.

The use of an external reference like the one that we present here cannot compensate for biological differences among samples nor for differences related to the quality of the chromatin extracts. However, the DNA-DIG-antibody complex restores variations that are related to the efficiency of the pull-down, to the reversal of the cross-linking and to the yield of recovery in the DNA purification. Reducing the variability of technical origin gives more consistent datasets that can reveal the biological differences of interest.

The method that we present here is universal because it can be used in conjunction with chromatin from any source. The DNA-DIG-reagent was designed to work in experiments that involve chromatin of animal origin. The specific DNA sequence that we have used does not have homologues in eukaryotes and the primers used to quantify it do not amplify any DNA sequences in samples from human or fly. DIG was the labeling of choice because DIG is not present in animals and because of the low cross-reactivity of the anti-DIG antibody in animal tissues. ChIP experiments with chromatin from other sources might require the use of alternative DNA sequences or the use of alternative labels to avoid cross-reactivity artifacts. The design of alternative probes more adequate to other organisms should be an easy task that only requires the use of basic bioinformatic tools. The large variety of antibodies that are commercially available makes it easy to choose alternative labels when necessary.

### REFERENCES

Gilbert C, Svejstrup J Q (2006) RNA immunoprecipitation for determining RNA protein associations in vivo. Curr Protoc Mol Biol, Chapter 27: Unit 27.4.
Aparicio O, Geisberg J V, Struhl K (2004) Chromatin immunoprecipitation for determining the association of proteins with specific genomic sequences in vivo. Curr Protoc Cell Biol, Chapter 17, Unit 17.7.
Listerman I, Sapra A K, Neugebauer K M (2006) Cotranscriptional coupling of splicing factor recruitment and precursor messenger RNA splicing in mammalian cells. Nat Struct Mol Biol 13: 815-822.
Barski A, Zhao K (2009) Genomic location analysis by ChIP-Seq. J Cell Biochem 107: 11-18.
Frericks H L, Zhou D H, Yap L L, Gennis R B, Rienstra C M (2006) Magic-angle spinning solid-state NMR of a 144 kDa membrane protein complex: E. coli cytochrome bo3 oxidase. J Biomol NMR 36(1):55-71.
Takahashi Y, Rayman J B, Dynlacht B D (2000) Analysis of promoter binding by the E2F and pRB families in vivo: distinct E2F proteins mediate activation and repression. Genes Dev 14: 804-816.

1. A method for conducting chromatin immunoprecipitation on a sample to be analyzed, comprising the steps of
a) providing a synthetic nucleic acid-antibody complex; and
b) adding the synthetic nucleic acid-antibody complex to the sample prior to immunoprecipitation.

2. The method according to claim 1, wherein said synthetic nucleic acid is DNA.

3. The method according to claim 1, wherein said synthetic nucleic acid is RNA, DNA-RNA hybrid, locked nucleic acid or peptide nucleic acid.

4. The method according to claim 1, wherein the synthetic nucleic acid is labeled with digoxigenin, biotin and/or fluorescein isothiocyanate.

5. The method according to claim 1, wherein said nucleic acid-antibody complex is formed by labeling a nucleic acid with a label, said label being recognizable by an antibody, cross-linking said labeled nucleic acid to an antibody recognizing said label, thereby forming said nucleic acid-antibody complex.

6. The method according to claim 5, wherein said cross-linking is performed by using formaldehyde or 3′-dithiobispropionimidate.

7. (canceled)

8. A method for preparing a nucleic acid-antibody complex according to claim 1, said method comprising the steps of:
a) providing a nucleic acid fragment;
b) label said nucleic acid fragment with a label to provide a labeled nucleic acid;
c) contacting said labeled nucleic acid of step b) with an antibody recognizing said label; and
d) cross-linking said antibody to said labeled nucleic acid to form a crosslinked antibody-nucleic acid complex.

9. A chromatin immunoprecipitation method comprising the steps of:
a) providing a cross-linked chromatin sample to be analyzed;
b) adding a synthetic nucleic acid-antibody complex according to claim 1 to the chromatin sample of step a);
c) performing an immunoprecipitation in the presence of an antibody recognizing a putative protein, such as histone, in the chromatin sample of step a);
d) reversing the cross-linking of said cross-linked chromatin sample and said nucleic acid-antibody complex;
e) purifying the DNA and nucleic acid obtained in step d); and
f) quantifying the amount of said DNA and said nucleic acid obtained in step e).

10. A kit of parts for use as an external standard in a chromatin immunoprecipitation method, said kit comprising:
a) a nucleic acid-antibody complex according to claim 1; and
b) a pair of primers to quantify the nucleic acid in the nucleic acid-antibody complex of a).

11. (canceled)

12. A kit of parts for performing a chromatin immunoprecipitation method, said kit comprising:
a) cell resuspension buffer;
b) glycine solution;
c) lysis and extraction buffers;
d) chromatin shearing buffer;
e) supplementary detergent solution for immunoprecipitation buffer;
f) washing buffer;
g) protease inhibitor cocktail;
h) pre-clearing beads;
i) pre-blocked Protein A or Protein G beads;
j) reversal solution;
k) DNA purification system;
l) external standard reagent; and
m) a synthetic nucleic acid-antibody complex.

13. A kit according to claim 12, further comprising peR primers for quantification of the external standard.

14. The kit according to claim 12, wherein said synthetic nucleic acid is DNA.

15. The kit according to claim 12, wherein said synthetic nucleic acid is RNA, DNA-RNA hybrid, locked nucleic acid or peptide nucleic acid.

16. The kit according to claim 12, wherein the synthetic nucleic acid is labeled with digoxigenin, biotin and/or fluorescein isothiocyanate.

17. The kit according to claim 12, wherein said nucleic acid-antibody complex is formed by labeling a nucleic acid with a label, said label being recognizable by an antibody, cross-linking said labeled nucleic acid to an antibody recognizing said label, thereby forming said nucleic acid-antibody complex.

18. The kit according to claim 17, wherein said cross-linking is performed by using formaldehyde or 3′-dithiobispropionimidate.
